(19) United States
(12) Patent Application Publication
(10) Pub. No.: US 2013/0006051 A1
(43) Pub. Date: Jan. 3, 2013

(54) VIDEO APPARATUS

(75) Inventors: Ian Douglas Stace, Minster-in-Thanet (GB); Natalie Lomas-Bullivant, Staplehurst (GB)

(73) Assignee: SMITHS MEDICAL INTERNATIONAL LIMITED, Ashford, Kent (GB)

(21) Appl. No.: 13/583,936

(22) PCT Filed: Mar. 17, 2011
(86) PCT No.: PCT/GB2011/000374

§ 371 Date: Sep. 11, 2012

(30) Foreign Application Priority Data
Apr. 15, 2010 (GB) 1006238.8

(51) Int. Cl. A61B 1/04

(52) U.S. Cl. 600/109

(57) ABSTRACT

Video apparatus, particularly for use in intubation of an endotracheal tube, has a bendable rod (10) with a camera unit (11) mounted at its forward, patient end (12). The camera unit (11) is retained by a sleeve (30) with at least a transparent window at its forward end (31). The sleeve (30) extends along the camera unit (11) and is bonded to the outside of the rod (10) at its forward end (12). The rod (10) has a bore (17) extending along its length through which a cable (24) from the camera unit (11) extends to a low profile connector (13) at the rear, machine end (14) of the rod.

This invention relates to video apparatus of the kind including a rod and a camera unit mounted at the forward end of the rod.

The invention is more particularly, but not exclusively, concerned with video apparatus of the kind used for aiding insertion of a tube into a body space, such as the insertion of an endotracheal tube into the trachea.

Traditional introducers take the form of a simple rod that can be bent to an approximate desired shape and flex to accommodate the shape of the anatomy during insertion. The introducer may be made with an angled, Coudé tip to facilitate introduction. The introducer can be inserted more easily than the tube itself because it has a smaller width and can be bent and flex to the ideal shape for insertion. The small width also gives the clinician a better view of the trachea around the outside of the introducer. When the introducer has been correctly inserted, a tube can be slid along the outside of the introducer to the correct location, after which the introducer is pulled out of the tube, which is left in position. Preferably the introducer only takes the desired shape temporarily and returns to its original shape after insertion, so that the introducer can be removed easily from the tube without disturbing it. Also, the introducer is preferably softened by the heat of the body, thereby making removal easier. Introducers are available from Smiths Medical. GB2312378 describes an introducer moulded of an aliphatic polyurethane material and also describes an earlier introducer made from a braided polyester filament repeatedly coated in layers of resin.

More recently it has been proposed to use fibre optics or a CCD camera with an introducer to provide the clinician with a view of the trachea as the introducer is inserted. WO2007/089491 describes an arrangement with an introducer and a separate camera assembly clipped onto the outside of the introducer, which is removed before an endotracheal tube can be slid along the introducer. Alternative arrangements have a handle at the machine end of the introducer so that the apparatus has to be inserted with the tube already loaded on the introducer from its patient end. Such arrangements are more difficult to use because of the presence of the tube during insertion. WO2010/136748 describes a video bougie that enables a tube to be slid along the bougie over its rear end.

One problem with such previous arrangements is the difficulty of securing the video camera and any light source at the patient end of the rod or bougie in a secure manner and at low cost. It will be appreciated that it is very important that the camera does not become detached from the rod during use. It is also important, particularly in disposable devices, that the attachment mechanism and manufacturing procedure does not add excessively to the overall cost of the device.

It is an object of the present invention to provide alternative video apparatus.

According to one aspect of the present invention there is provided video apparatus of the above-specified kind, characterised in that the apparatus includes a sleeve enclosing the camera unit with the forward end of the sleeve being located at the forward end of the camera unit and providing a transparent viewing window for the camera unit, and that the sleeve extends rearwardly beyond the rear end of the camera unit along the forward end of the rod and is secured thereto.

The rod preferably includes a passage along its length and the camera unit preferably includes a cable extending rearwardly along the passage and connected to a connector mounted at the rear end of the rod. The camera unit preferably includes illumination means arranged to illuminate the field of view of a camera in the unit. The rear end of the sleeve is preferably bonded to the rod by means of an adhesive. The camera unit may be butted against the forward end of the rod. The forward end of the rod may be formed with a step and a recess around its outside with a depth substantially equal to the thickness of the rear end of the sleeve such that the sleeve can be secured in the recess up to the step and form a substantially smooth join between the outside surface of the sleeve and the rod.

According to another aspect of the present invention there is provided a method of manufacturing video apparatus including the steps of providing an elongate rod and a separate camera unit having a viewing end, a side and a rear end, enclosing the camera unit in a sleeve with a transparent viewing window extending over the viewing end of the camera unit and with the sleeve extending along the side of the unit to project beyond the rear end of the camera unit, and extending the projecting end of the sleeve along the forward end of the rod such that the sleeve retains the camera unit on the rod.

The sleeve is preferably bonded to the rod by means of a solvent, adhesive or weld.

According to a further aspect of the present invention there is provided video apparatus made according to the method of the above other aspect of the present invention.

Video apparatus according to the present invention in the form of a video bougie, will now be described, by way of example, with reference to the accompanying drawings, in which:

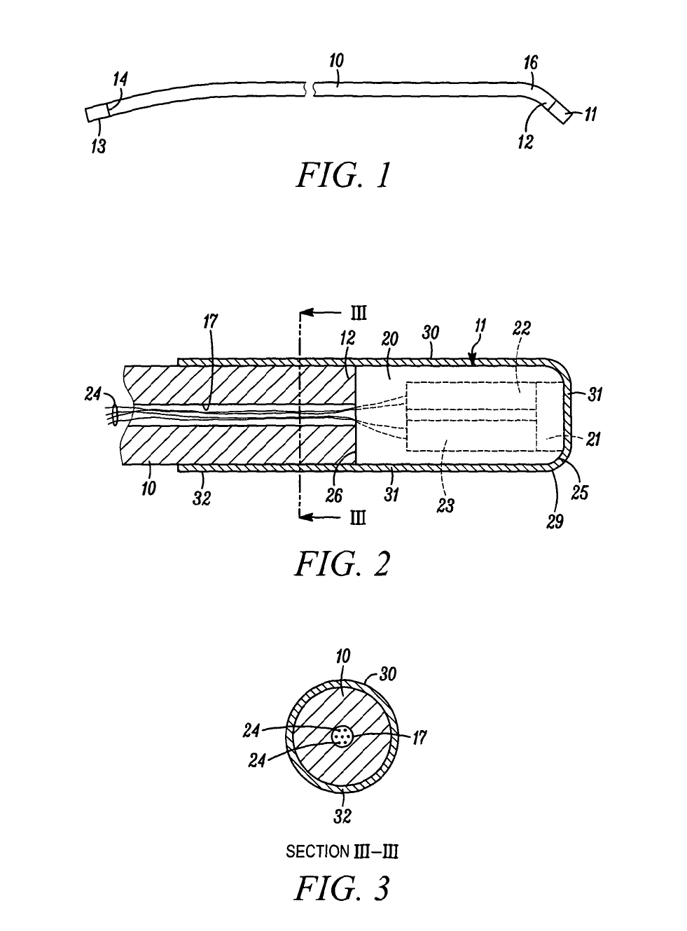

FIG. 1 is a side elevation view of the video bougie;

FIG. 2 is an enlarged cross-sectional side elevation view of the forward end of the video bougie;

FIG. 3 is an enlarged cross-sectional transverse view along the line III-III of FIG. 2;

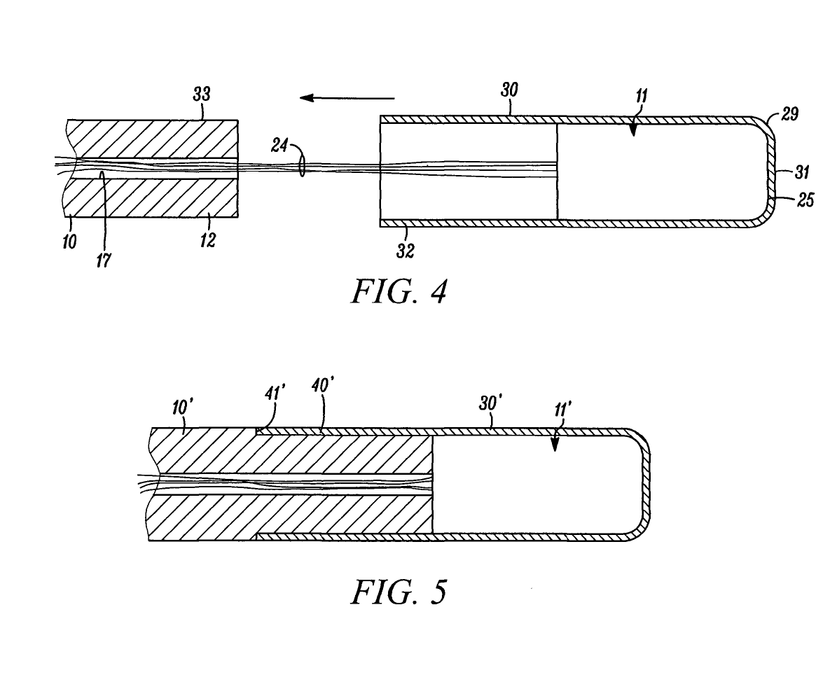

FIG. 4 shows the forward end of the apparatus at a preliminary stage of manufacture;

FIG. 5 is an enlarged cross-sectional side elevation view of the forward end of a modified apparatus; and

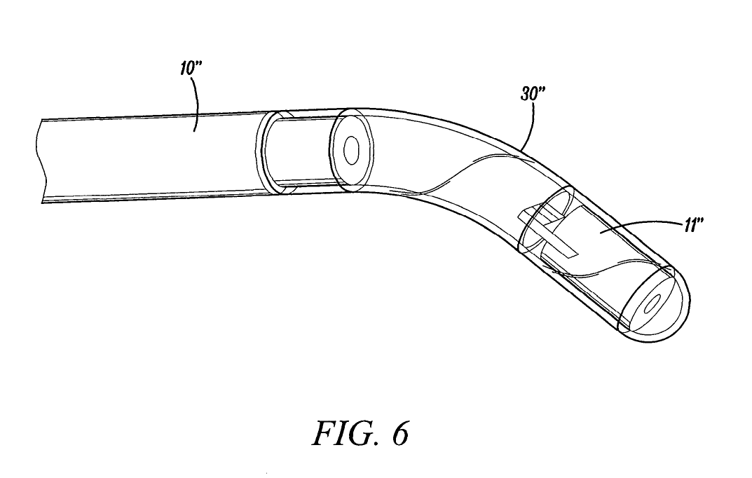

FIG. 6 is an enlarged perspective view of the forward end of another modified apparatus.

With reference first to FIG. 1, the apparatus is in the form of a bougie or introducer 1 comprising a rod 10, a camera unit 11 mounted at the forward, patient end 12 of the rod, and an electrical connector 13 mounted at the rear end 14 of the rod. The connector 3 is connected to a video display (not shown) in the manner described in WO2010/136748.

The rod 10 is similar to that used in conventional endotracheal introducers or bougies, being about 720 mm long (about twice the length of a typical endotracheal tube) with a circular section about 5 mm in diameter, although other sizes could be used. In its natural state the rod 10 is straight apart form a short, preformed Coudé tip portion 16 at its patient end 12 extending at an angle of about 40° to the remainder of the rod. The rod 10 is made of a PVC material by extruding and then heat forming the Coudé tip 16, although other techniques could be used such as injection moulding. The rod 10 is formed with a narrow bore 17 (FIG. 2) along its length.

With reference now also to FIGS. 2 and 3, the camera unit 11 is formed separately of the rod 10 comprising a moulded, generally cylindrical housing 20 with a slightly oval section being 3 mm wide and 4 mm high. The unit 11 has an opening or a transparent window 21 at its patient end. The camera unit 11 has approximately the same width as the rod 10 and is formed with a rounded, atraumatic forward, patient end 25 and a flat rear end 26 of the same shape and size as the forward end of the rod 10. The unit 11 is about 8 to 15 mm long. The housing 20 contains a CCD video camera sensor 22 and one or more white-light LEDs 23 mounted below the camera lens. It will be appreciated that the video camera sensor 22 may include conventional drive and processing circuitry. The camera 22 and LEDs 23 are directed axially forwardly through the window 21 and are electrically connected via a flexible cable or wires 24, which extend through the bore 17 of the rod 10 and connect to the electrical connector 13. The flexibility of the wires 24 is such that they do not adversely affect the handling of the introducer 1.

The camera unit 11 is secured to the forward end of the rod 10 mainly by means of a sleeve 30. The sleeve 30 is injection moulded from a transparent plastics material, which may be rigid, such as of Cyrolite G20, or flexible, such as a polyurethane, for example Tecoflex. The sleeve 30 is formed of a cylindrical shape with a wall 31 having a thickness of about 0.2 mm and an internal diameter approximately equal to the external dimensions of the camera unit 11. The sleeve 30 is closed at its forward end 29 by means of an integral window 31 curved to the rounded shape of the forward end 25 of the camera unit 11. The sleeve 30 is about twice the length of the camera unit 11. The sleeve 30 is moulded separately of the camera unit 11 and is fitted onto the camera unit as a close fit with the window 31 of the sleeve lying against the opening or window 21 of the camera unit. Where the camera unit 11 has a solid transparent window 21 it may be advantageous to coat the outside of the window with a liquid or gel having a refractive index matched to that of the window in the camera unit and the sleeve 30 so as to improve light transmission. The rear end 32 of the sleeve 30 extends rearwardly beyond the rear of the camera unit 11 along the outside of the forward end of the rod 10. A uv adhesive 33 is applied between the outside of the rod 10 and the sleeve 30 to bond the sleeve to the rod. It will be appreciated that other techniques could be used to bond the sleeve 30 to the rod 10, such as ultrasonic welding or a solvent bond.

The sleeve 30 serves a dual purpose of retaining the camera unit 11 on the end of the rod 10 and also providing a liquid-tight seal around the camera unit. The wires 24 also provide some additional security of retention in the event that the sleeve 30 should be damaged. The sleeve 30 enables the maximum width of the apparatus to be utilised compared with previous arrangements where the camera and illumination means is enclosed within a cavity in the rod itself The wall of the camera housing 20 can be made relatively thin, enabling a smaller external overall diameter or a larger, high resolution camera sensor 22 or more illumination LEDs 23 to be employed.

The apparatus is assembled in the manner shown in FIG. 4. The preformed sleeve 30 is first slipped onto the camera unit 11 so that the closed end 31 of the sleeve abuts the forward end 25 of the camera unit. The rounded forward end 25 of the camera unit 11 ensures that the unit does not have sharp edges that could damage the sleeve 30. The wires 24 extending rearwardly from the camera unit 11 are threaded along the bore 17 of the rod 10 to emerge from its rear end. The uv adhesive 33 is painted onto the outside of the forward end 12 of the rod 10, the rear end of the sleeve 30 being slid along the rod until the rear of the camera unit 11 abuts the forward end of the rod. The adhesive 33 lubricates and helps the sleeve 30 slip along the rod 10. As the sleeve 30 is slid rearwardly, the wires 24 are pulled gently out of the rear end of the rod 10. When the sleeve 30 has been fully extended onto the rod 10, the join is exposed to uv light to cure the adhesive 33 and form a secure bond between the sleeve and the rod. The wires 24 are subsequently attached to the connector 13.

Instead of preforming the sleeve and subsequently assembling it on the camera unit 11, it might be possible to mould the sleeve directly onto the camera unit and thereby avoid the step of assembling the sleeve onto the camera unit.

FIG. 5 illustrates a modification to the apparatus described above in which equivalent components have been given the same number with the addition of a prime ′. The camera unit 11′ and sleeve 30′ are identical to those shown in FIGS. 1 to 4 except that the external width of the camera unit is 0.4 mm less than that of the rod 10′. The forward end of the rod 10′ is modified by removing (such as by machining) a thin surface portion from the outside of the rod to form a shallow annular recess 40′ and a step 41′. The depth of the recess 40′ is 0.2 mm, that is, equal to the thickness of the sleeve 30′, and the length of the recess is equal to that of the projecting rear end of the sleeve 30′ so that the sleeve extends along the length of the recess and abuts the step 41′. This arrangement ensures a smooth external surface to the completed apparatus by avoiding any external step.

FIG. 6 illustrates another modification of the apparatus described above in which equivalent components have been given the same number with the addition of a double prime “. The sleeve 30” is longer than the sleeves 30 and 30′ in the other embodiments, being about 25 mm long and is preformed with a bend along its length. The sleeve 30″ is made of a rigid material, such as Cryolite G20 so that it substantially retains its curved shape. In this way, the sleeve 30″ itself forms the Coudé tip for the apparatus enabling the rod 10″ to be formed straight.

It will be appreciated that the apparatus could be modified in various different ways. The sleeve need not be entirely transparent providing that it has at least a transparent window at its forward end, which could be provided by a transparent material or by an aperture. Where the sleeve has an aperture this is preferably filled with a transparent potting material. The dimensions and shape of the apparatus could be modified for various different applications. The rod could be rigid or flexible depending on the application. Instead of having wires extending from the camera unit the output could be provided by wireless means, such as by radio signals. In this case the camera unit would include a battery or some other form of power source to power the camera and illumination means. Instead of using a light-emitting element, such as an LED, the introducer could include a fibre optic cable extending along the bore of the rod and connected to a light source in the display unit via a hybrid fibre optic/electrical connector at the machine end of the rod. The invention is not limited to devices for inserting a tracheal tube but could find application in other apparatus where a camera needs to be mounted at the end of a rod or elongate member.

1-9. (canceled)

10. Video apparatus including a rod and a camera unit mounted at the forward end of the rod, characterized in that the apparatus includes a sleeve of a transparent material enclosing the camera unit with the forward end of the sleeve being located at the forward end of the camera unit and providing a transparent viewing window for the camera unit, and that the sleeve extends rearwardly beyond the rear end of the camera unit along the forward end of the rod and is secured thereto.

11. Video apparatus according to claim 10, characterized in that the rod includes a passage extending along its length, and that the camera unit includes a cable extending rearwardly along the passage and connected to a connector at the rear end of the rod.

12. Video apparatus according to claim 10, characterized in that the camera unit includes illumination means arranged to illuminate the field of view of a camera in the unit.

13. Video apparatus according to claim 10, characterized in that the rear end of the sleeve is bonded to the rod by means of an adhesive.

14. Video apparatus according to claim 10, characterized in that the camera unit is butted against the forward end of the rod.

15. Video apparatus according to claim 10, characterized in that the forward end of the rod is formed with a step and a recess around its outside with a depth substantially equal to the thickness of the rear end of the sleeve such that the sleeve can be secured in the recess up to the step and form a substantially smooth join between the outside surface of the sleeve and the rod.

16. A method of manufacturing video apparatus including the steps of providing an elongate rod and a separate camera unit having a viewing end, a side and a rear end, enclosing the camera unit in a sleeve of a transparent material with a transparent viewing window extending over the viewing end of the camera unit and with the sleeve extending along the side of the unit to project beyond the rear end of the camera unit, and extending the projecting end of the sleeve along the forward end of the rod such that the sleeve retains the camera unit on the rod.

17. A method according to claim 16, characterized in that the sleeve is bonded to the rod by means of a solvent, adhesive or weld.

18. Video apparatus made by a method according to claim 16.
